(19) United States
(12) Patent Application Publication
(10) Pub. No.: US 2003/0216784 A1
(43) Pub. Date: Nov. 20, 2003

(54) Cellular electromanipulation waveforms

(76) Inventors: Richard Heller, Temple Terrace, FL (US); Richard Gilbert, Temple Terrace, FL (US); Mark J. Jaroszeski, Wesley Chapel, FL (US)

Correspondence Address: SMITH & HOPEN PA, 15950 BAY VISTA DRIVE, SUITE 220, CLEARWATER, FL, 33760

(21) Appl. No.: 10/064,660

(22) Filed: Aug. 5, 2002

Related U.S. Application Data
(63) Continuation-in-part of application No. 09/507,859, filed on Feb. 22, 2000.

(51) Int. Cl. A61N 1/18

(52) U.S. Cl. 607/3

(57) ABSTRACT

The present invention is a method of electromanipulation for effecting substantially simultaneous electroporation and electromigration of molecules into cells by applying to a cellular target a preselected electrical waveform. The preselected electrical waveform may be formed of at least one curved or linear component either increasing or decreasing in amplitude as a function of time. In a preferred embodiment of the invention the at least one component has a duration no greater than five minutes and a maximum amplitude no greater than 10,000 V/cm. Alternatively, the waveform may also include a substantially constant amplitude component interposed between the increasing and decreasing components. The substantially constant amplitude component may also be applied prior or subsequent to the at least one component.

### CROSS REFERENCE TO RELATED APPLICATION

This application claims the benefit and is a Continuation-in-Part of U.S. patent application Ser. No. 09/507,859 filed Feb. 22, 2000. The disclosure of the previous application is incorporated herein in its entirety by reference.

### BACKGROUND OF INVENTION

1. Field of the Invention

This invention relates to a method and apparatus for delivering molecules into cells, inducing cells to fuse, inducing cells to fuse to tissue, and moving molecules across a living or dead cellular barrier by deploying novel electrical waveforms.

2. Background of the Invention

The effect of electricity on the membranes of living cells has been under investigation since the 1960's and 1970's. Early research was focused on describing observations that an applied electric field can reversibly break down cell membranes in vitro. Throughout the 1970's the topic was more common in the literature and continued to focus on describing the phenomenon that resulted from brief exposure to intense electric fields as well as the entry of exogenous molecules to the cell interior as a result of membrane breakdown. Applications for this technology began to emerge in the 1980's.

Research has lead to the current understanding that the exposure of cells to intense electric fields for brief periods of time temporarily destabilizes cell membranes. This destabilization has been described as a dielectric breakdown due to an induced transmembrane potential that results from electrical treatment. This physical phenomenon was termed electroporation, or electropermeabilization, because it was observed that molecules that do not normally pass through the membrane can gain intracellular access after the cells were treated with electric fields. The porated state was noted to be temporary. Typically, cells remain in a destabilized state on the order of minutes after electrical treatment ceases. The physical nature of electroporation makes it universally applicable. A variety of procedures utilize this type of treatment which temporarily destabilizes the membrane including the delivery of molecules to the interior of cells in vitro, delivery of molecules to cells comprising tissues in vivo and ex vivo, cell-cell fusion, and cell-tissue fusion in vitro and in vivo. In addition, applications that involve the use of electric fields to transport molecules across the stratum corneum also use electric fields.

Typically a molecule of interest is administered before electrical treatment so that it is present in the vicinity of cell membrane when electroporation occurs and can then diffuse, migrate, or be electrically translocated through the permeabilized membrane of a cell or through the a layer of cells.

Electric fields have been applied to cells/tissue for the purposes described above as a voltage driven waveform or series of voltage driven waveforms These series are often referred to as trains of waveforms, and a common feature of all trains is that there is a time period between each waveform where the applied field/voltage is zero. The vast majority of the waveforms used are rectangular direct current waves or exponentially decreasing direct current waveforms. Typically one or more discrete and identical waveforms are administered using custom made or commercially available electrical generators. Custom made and commercially available electrodes are also used as a means for interfacing the electrical generators to the cells/tissue under treatment.

It is commonly accepted in the art that all biological entities bound by membranes can be electroporated by exposure to electric fields. It is also commonly accepted that there is a threshold for the applied field that is required to induce membrane destabilization. Typically, electric-fields that are below this threshold are not sufficient to cause membrane breakdown and those that are above it are sufficient to induce membrane effects. The threshold for any given membrane entity may vary. For example, it is known that smaller cells require a higher applied electric field for electropermeabilization and that larger cells require a lower applied electric field. Other variables that influence this dependence may be particular to the biology of the cell and environment that the electrical treatment is conducted in.

Electrical treatment is described by those in the field by indicating the electric field strength of the applied waveforms, number of waveforms administered, the interval between successive waveforms in series, the shape of each waveform, and an indicator of the duration of each waveform. A broad range values for each of these variables have been used; these ranges are similar for performing electroporation, electrofusion, and for delivering molecules across layers of cells.

The electric field strength applied is described based on the distance between the electrodes used to apply the fields. Values ranging from approximately 100 to 6,000 V/cm have been used. This number can be normally be interpreted as the maximum applied field. The number of successive waveforms administered has ranged from approximately 1 to 8; these are typically identical waveforms. Normally there is a time interval between successive waveforms that is sometimes designated as the time between the initiation of each waveform and in other similar ways. However, regardless of the manner in which the description is made the values usually fall somewhere in the milliseconds to seconds time frame.

As indicated previously, two primary waveforms are commonly used in the prior art. These are rectangular direct current and exponentially decaying. Rectangular waveforms have a profile in time that includes a very rapid increase from a field of 0 V/cm to a predetermined value, a period of substantially constant electric field application, and then a rapid decline back to 0 V/cm. Increases and decreases in rectangular waveforms are as close to instantaneous as current state of the art electronics can achieve. The duration of the constant electric field, or duration, normally falls within the range of 10 microseconds to 100 milliseconds. Exponentially decaying waveforms typically have a shape that is characterized by a rapid increase from 0 V/cm to a predetermined value that immediately begins decreasing in an exponential manner until a value of 0 V/cm is reached. These waveforms are generally created by the discharge of an electrical capacitor. The descriptive term used for the duration of exponentially decreasing waveforms is sometimes an RC constant (the time constant of a series RC circuit is the product of the resistance value times the capacitance value where the time constant is in seconds, the resistance is in ohms and the capacitances is in farads) and is sometimes the time between the start of the waveform until it has decayed substantially to 0 V/cm. Exponentially decaying pulses that require microseconds to milliseconds to decay from initiation to substantially 0 V/cm have been used.

In an article by Sukharev et. al entitled: “Electroporation and electrophoretic DNA transfer into cells” in Biophys. J., Volume 63, November 1992, pages 1320-1327, there is a disclosure two voltage driven generators were employed to administer two waveforms that had the same shape but were different in field strength and duration to Cos-1 cells in order to transfer plasmid DNA coding for the beta galactosidase gene through the cell membranes and into the cells. The waveforms administered were designed to perform two distinct functions when applied to the cells. The first single waveform was designed to cause membrane breakdown, or electroporation. The magnitude of this waveform ranged from 4-7 kV/cm and was 10-20 microseconds in duration; waveforms within these-parameter ranges were determined to induce electroporation in the cells. The second waveform was designed to cause electromigration of the DNA into the electroporated cell membrane. The second waveform had much lower range of field strengths, 0.2-0.4 kV/cm, and a longer range of durations, 10-20 milliseconds, than the waveform that were administered first. The second waveforms were determined not to cause any detectable transfection of the foreign DNA. The interval between administration of the two discrete waveforms ranged from 100 microseconds to 100 seconds.

Results of the Sukharev et. al study indicated that administering a single first high field strength waveform (as described above) that was sufficient to induce electroporation followed by a single second lower field strength waveform (also as described above) resulted in a higher transfection efficiency that when either single waveform was used alone. The study teaches that an electromigration effect of the second pulse was responsible for moving exogenous DNA through the cell membranes and into the cells. This study applied the first and second pulses as a concatenated series with a time interval between the pulses, thus separating them and their functions as discrete events. The study results clearly showed that decreasing the time interval between the two waveforms from 100 seconds to 100 microseconds lead to increased transfection efficiencies. This study only addressed the use of a total of one waveform for electroporation followed by one waveform for electromigration.

In an article by Andreason et al entitled: “Optimization of electroporation for transfection of mammalian cell lines” in Anal. Biochem., Vol.180, No. 2, pages 269-275, 1, 1989, there is a disclosure that applying waveforms that have different functions is advantageous for transferring DNA to cells compared to applying a waveform intended only to electroporate or cause electromigration. This study used a single rectangular waveform that was 1,000 to 3,000 kV in field strength and 15 microseconds in duration to induce electroporation. This was followed by a series of 12 rectangular waveforms that were 24-34 V/cm in field strength and 20 microseconds in duration. This series of waveforms were administered with at intervals of 2 seconds. No explicit mention of the time interval between the administration of the first higher field strength waveform and the series of lower field strength waveforms was made, but the study always refers to them as discrete events.

The study found that the series of lower field strength waveforms following the higher field strength waveform lead to markedly increased transfection efficiencies relative to using the high field strength waveform alone for DNA delivery. The study did not explicitly mention electromigration of DNA as a mechanism by which the series of low field strength waveforms improved transfection efficiency.

A study by Teissie entitled: “Time Course of Electropermeabilization” in Charge and Field Effects in Biosystems-3 (Allen, M J., Cleary, S. F., Sowers, A. E., and Shillady, D. O. eds.) Birkhauser, Boston, Mass., Pp. 285-301. indicates that electroporation is a cellular event that has a lifetime that is on the order of minutes. The study also indicated that the ability to transport molecules through cells that have been treated with electroporating pulses decreases starting at the time that the application of energy ceases until the cells reseal over the course of several minutes.

In an article by Okamoto et al entitled: “Optimization of Electroporation for Transfection of Human Fibroblast Cell Lines with Origin-Defective SV40 DNA: Development of Human Transformed Fibroblast Cell Lines with Mucopolysaccharidoses (I-VII)” in Cell Structure and Function, Vol.17, (1992), pages 123-128, there is a disclosure that a variety of variables relative to rectangular waveforms for electroporation. The electric parameters included voltage (field strength), pulse duration, number of pulses, and pulse shape. All pulses were of the same duration, magnitude, and pulse interval when a series of waveforms were administered.

In an article by Ohno-Shosaku et al entitled: “Somatic Hybridization between Human and Mouse Lymphoblast Cells Produced by an Electric Pulse-induced Fusion Technique” in Cell Structure and Function, Vol. 9, (1984), pages 193-196, there is a disclosure of the use of an alternating electric field of 0.8 kV/cm at 100 kHz to fuse biological cells together. It is noted that the alternating current provides a series of electrical pulses all of which have the same duration, the same magnitude, and the same interval between pulses.

A manuscript by Zheng and Chang entitled: “High-efficiency gene transfection by in-situ electroporation of cultured cells” in Biochimica et. Biophisica Acta. 1088: 104-110 (1991) discloses a electroporation using alternating current biased with a constant direct current signal. This waveform was administered to the cells as a series of 5 biased alternating current cycles followed by an time period 1 second when no voltage was applied. This sequence was repeated.

Throughout the history of the field relating to the effects of electric fields on cells it is clear that a total of only three distinct waveforms have been utilized. These are rectangular waveforms, exponentially decaying waveforms, and direct current biased sinusoidal alternating current waveforms.

U.S. Pat. No. 6,010,613 advanced the art by providing a method for electrically treating organic material and biological cells with a sequence of at least three singular waveforms that are rectangular where (1) at least two of at least three waveforms differ from each other in pulse amplitude; (2) at least two of the at least three waveforms differ from each other in width (duration); and (3) a first waveform interval for a first set of two of the at least three waveforms is different from a second waveform interval for a second set of two of the at least three waveforms. The '613 reference maintained that the viability of cells can be maintained and that the lifetime of electropores can be prolonged by applying electric fields. All of the waveforms described in the '613 reference are discrete and separated by an interval of time where the applied field is 0 V/cm and makes reference to rectangular waveforms and the waveform generator design criteria in the specification also teach that rectangular waveforms be applied.

Thus, while the foregoing body of prior art indicates that it is well known to use electrical pulses to induce the breakdown of cell membranes for a variety of purposes including electroporation, prolonging the electroporated state, and causing the electromigration of molecules. The prior art described above does not, however, teach or suggest a method for treating biological materials that contain membranes with waveforms that are single and continuous with components that can induce electroporation, induce molecule migration, and sustain the electroporated state.

Accordingly, what is needed in the art is a method of applying a voltage for electromanipulation of a cell that achieves both electroporation and electromigration in a single waveform.

Another need in the art exists for a method of applying a voltage for electromanipulation of a cell that achieves cell-to-cell fusion and cell-tissue fusion in addition to electroporation and electromigration with a single waveform.

It is, therefore, to the effective resolution of the aforementioned problems and shortcomings of the prior art that the present invention is directed.

However, in view of the prior art in at the time the present invention was made, it was not obvious to those of ordinary skill in the pertinent art how the identified needs could be fulfilled.

### SUMMARY OF INVENTION

The present invention is a method of electromanipulation for effecting substantially simultaneous electroporation and electromigration of molecules into cells by applying to a cellular target a preselected electrical waveform. The preselected electrical waveform may be formed of at least one curved component either increasing or decreasing in amplitude as a function of time. The at least one curved component is defined to comprise any sinusoidal, harmonic, or exponential shape. In a preferred embodiment of the invention the at least one curved component has a duration no greater than five minutes and a maximum amplitude no greater than 10,000 V/cm. The preselected electrical waveform may include both increasing and decreasing curved components. Alternatively, the waveform may also include a substantially constant amplitude component interposed between the increasing and decreasing curved components. The substantially constant amplitude component may also be applied prior or subsequent to the at least one curved component.

An alternative to employing a curved shape is to use at least one linear component. It is preferred that the at least one linear component has a duration no greater than five minutes and a maximum amplitude no greater than 10,000 V/cm. The at least one linear component may increase or decrease in amplitude as a function of time. The waveform may also include a combination of increasing and decreasing linear components. A substantially constant amplitude component may be interposed between the increasing and decreasing linear components. The waveform may also include a substantially constant amplitude component interposed between the increasing and decreasing linear components. The substantially constant amplitude component may also be applied prior or subsequent to the at least one linear component.

In yet another alternative embodiment of the invention the preselected electrical waveform includes a plurality of coincident, substantially rectangular components whereby the latest time that the following rectangular component can begin is substantially simultaneously with the completion of the preceding rectangular component. The plurality of coincident, substantially rectangular components may be of similar or differing amplitudes. Preferably, the plurality of coincident, substantially rectangular components have durations no greater than five minutes and maximum amplitudes no greater than 10,000 V/cm.

It should also be noted that the preselected electrical waveform may have an amplitude less than 0 and also be administered in series forming a pulse wherein at least two preselected electrical waveforms in the pulse are of differing shape.

The novel application of these waveforms may be employed for effecting the membranes of living biological cells including, but not limited to (1) cells in an in vitro environment; (2) cells existing as components of a tissue in an in vitro, in situ, and in vivo environment; and (3) tissues in the body of a human or animal for the purpose of cell-cell fusion, cell-tissue fusion, causing the electromigration of molecules. The electromigration of molecules may include, but is not limited to, polynucleotides and drugs into and out from the cytoplasmic compartment of cells, and causing a transient breakdown in the cytoplasmic membranes of cells for the purpose of delivering molecules to the cell. This may be effectuated by applying electromagnetic energy using a suitable electrical generator and electrodes as known in the art.

It is therefore an object of the present invention to provide an improved method for effecting cell membranes for the purpose of electroporation of single cells and cells arranged as tissues both in vitro and in vivo, cell-cell electrofusion, and celltissue electrofusion both in vitro and in vivo.

It is an additional object of this invention to provide an improved method for causing the electromigration of molecules in the vicinity of cell membranes and through cell membranes both in vitro and in vivo.

It is yet another object of this invention to provide an improved method for causing the electromigration of molecules in the vicinity of cell membranes, through cell membranes, and into the interior of cells both in vitro and in vivo.

It is a further object of the present invention to provide an improved method for effecting the transport of molecules across layers of living or dead cells.

### BRIEF DESCRIPTION OF DRAWINGS

For a fuller understanding of the nature and objects of the invention, reference should be made to the following detailed description, taken in connection with the accompanying drawings, in which:

FIG. 1 is a diagrammatic illustration of the general principle of electroporation of a cell by application of a voltage.

FIG. 2 is a diagrammatic illustration of cell-cell electrofusion.

FIG. 3 is a waveform having symmetrical, exponentially rising and decreasing components.

FIG. 4 is a first embodiment of a waveform having non-symmetrical, exponentially rising and decreasing components.

FIG. 5 is a second embodiment of a waveform having non-symmetrical, exponentially rising and decreasing components.

FIG. 6A is a waveform that combines a period of constant amplitude between exponentially increasing and decreasing portions.

FIGS. 6B-D are waveforms having an exponentially increasing waveform combined with a constant amplitude waveform.

FIGS. 6E-F are waveforms that have exponentially decreasing components combined with a constant amplitude component.

FIGS. 7A-C show three embodiments of waveforms that use linearly increasing and decreasing waveforms.

FIGS. 7D-F show three embodiments of waveforms that use a single linearly increasing component alone or combined with a constant amplitude component.

FIGS. 7G-I show three embodiments of waveforms that comprise linearly decreasing components.

FIGS. 7J-L show three embodiments of waveforms with at least two regions of constant amplitude each.

FIG. 8A shows a waveform with an exponentially increasing, constant amplitude and linearly decreasing components combined into one continuous signal.

FIG. 8B shows an alternative embodiment of the waveform in FIG. 8A that includes a linearly increasing component followed by a constant amplitude component in one continuous form.

FIG. 8C shows a pulse comprised of a series of identical waveforms.

FIG. 8D shown a pulse comprising at least two distinct waveforms.

FIGS. 9A-B show pulses comprising waveforms of negative amplitudes.

FIGS. 10A-C show a series of waveforms applied to a quantitative study of waveforms disclosed in the present invention.

FIG. 11 shows the resulting mean luciferase expression for each of five animal groups according to a quantitative study of waveforms disclosed in the present invention.

### DETAILED DESCRIPTION

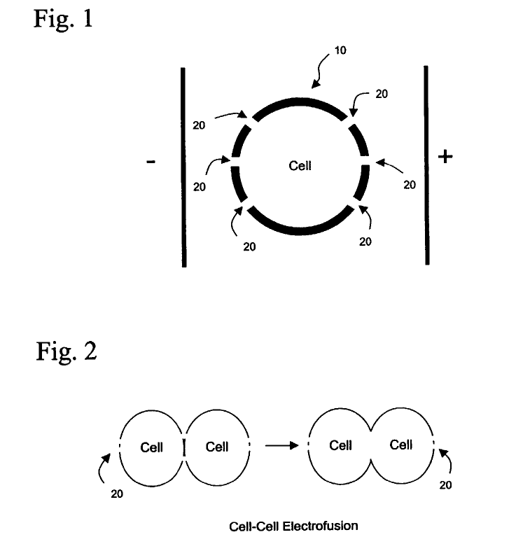

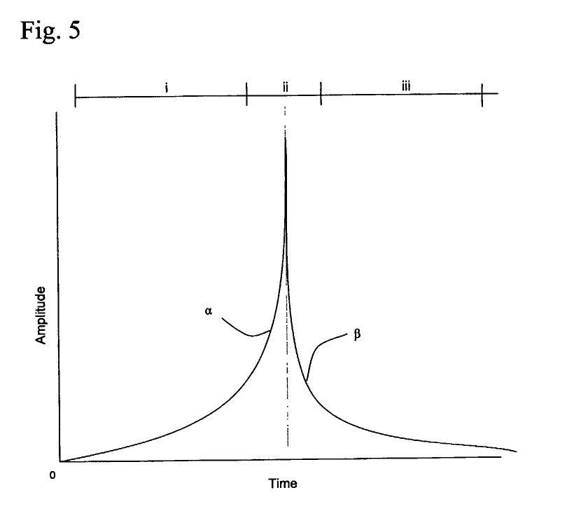

A description of the preferred embodiments of the present invention will now be presented with reference to FIGS. 1-11. It should be stressed that the waveforms presented can be administered with a voltage driven device or a current driven device.

FIG. 1 depicts the process of electroporation. This process has been used in vitro as well as in vivo and is typically carried out by first exposing the cells or tissue of interest to electric fields that are administered using an electrical generator and suitable electrodes. Electrical treatment is conducted in a manner that results in temporary membrane destabilization with minimal cytotoxicity. Destabilized areas in the membrane are referred to as electropores as indicated in the figure. Electropores have lifetimes that are on the order of minutes.

FIG. 2 depicts an additional effect of electric fields on living cells. Cells that have been electrically treated can fuse together. This occurs if the two electrically treated cells come in contact with each other before, during, or after the application of energy. A common lumen between two or more cells can be formed during the process of membrane resealing that takes place, generally, after electrical treatment has ceased. Cells are typically forced into contact using methods such as centrifugation, vacuum deposition, biochemicals, dielectrophoresis, and other means.

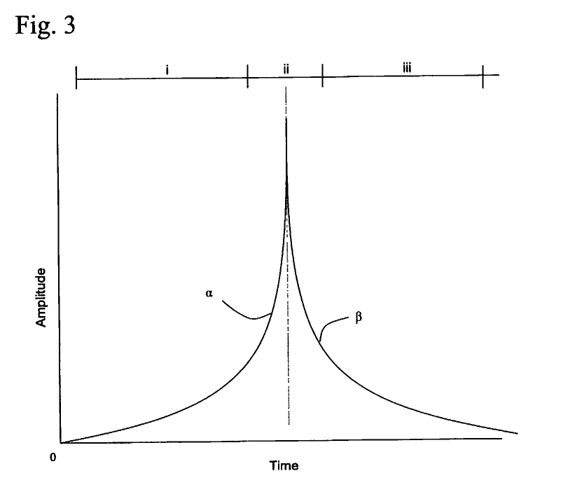

This invention is continuous electrical waveforms with defined shapes that can be used to effect the membranes of living biological cells in a manner that facilitates the delivery of molecules to the interior of living biological cells and to induce the fusion of cells to each other. FIG. 3 shows an example of this type of pulse that can physically be described in two different parts, α and β. Part a is an exponentially rising component and part β is an exponentially decreasing component. The curvature and slope of parts α and β can be of any substantially exponential shape. This type of waveform can be produced using currently available electronic devices. The labels i, ii, and iii indicate different functional parts of the waveform. Part i may serve to facilitate the movement of molecules to or near the surface of a living biological cell and/or throughout a tissue; it also serves to charge to membrane of a cell. Membrane charging may facilitate the attraction of molecules to the cell membrane and may also facilitate the attraction and contact of fusion partners. Part ii serves to induce a transmembrane potential in cells that is sufficient to induce the dielectric membrane breakdown known as electroporation (also known as electropermeabilization). When a cell is electroporated it is known that this is also a fusogenic state. Finally, part iii serves to move molecules from the exterior of the cells to the interior through the permeabilized membrane; it may also prolong the electroporated state by causing an ionic flow through electroporated membrane thereby inhibiting the fluid-like membrane from resealing which can increase molecular transport into the cell and prolong the fusogenic state. The peak voltage of the entire wave form can range from 0-10,000 volts per centimeter and the time of the entire pulse can range from 0-5 minutes.

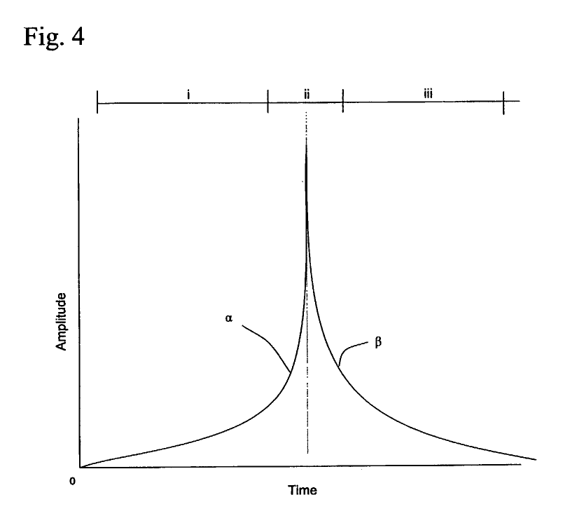

Part i and ii, of FIG. 3, have characteristics that are generally different from those of part iii. Parts i and ii are typically a lower field strength and longer time than part iii. It is not necessary for parts α and β to be symmetric. Both halves of the wave can be markedly different as shown in FIG. 4 without departing from the invention which is to induce molecular movement and electroporation from one substantially continuous wave form.

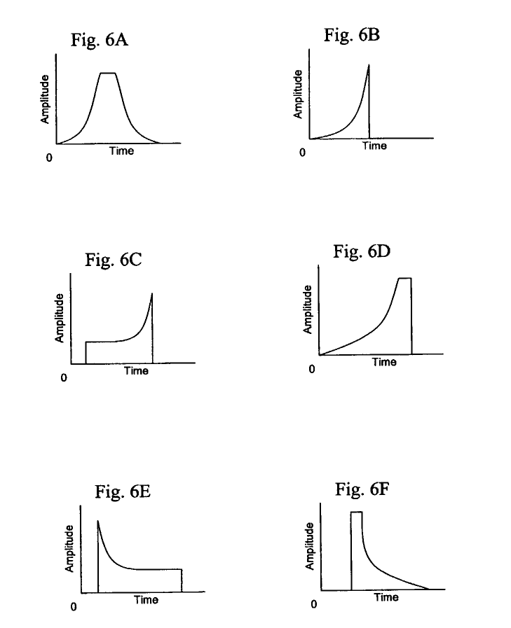

FIG. 6A shows a variation on the waves shown in FIGS. 3-5 that combines a period of constant amplitude that is incorporated between the exponentially increasing and exponentially decreasing portions of the waveform. FIGS. 6B-6D shows further embodiments that consist of an exponentially increasing waveform and this same type of waveform combined with a constant amplitude waveform to make a continuous waveform that provides an higher amplitude component for inducing electroporation and a lower amplitude region for inducing electromigration and/or prolonging the duration of the electroporated state. FIGS. 6E and 6F shows two embodiments of the invention that include waveforms that have an exponentially decreasing component and a constant amplitude component.

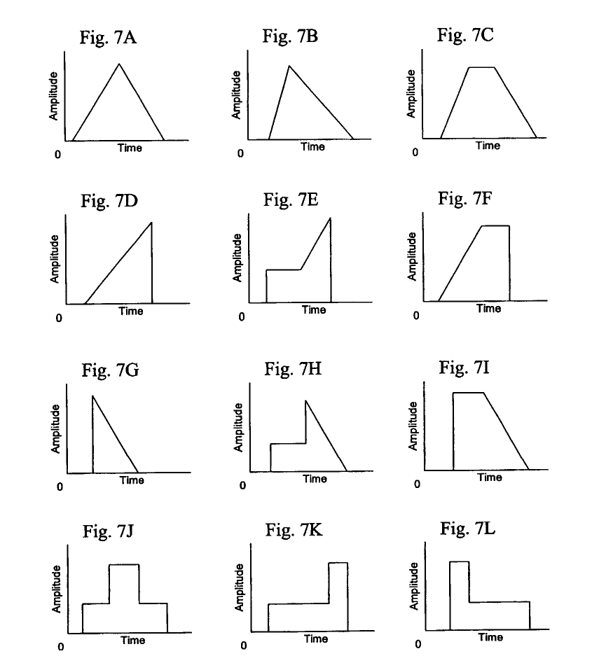

FIGS. 7A-7C indicate three embodiments that use linearly increasing and decreasing waveforms. FIG. 7A shows a waveform that is simply increasing and decreasing linear components that have equivalent absolute values of their slopes. FIG. 7B shows a similar waveform that has components with different absolute values of their slopes, and FIG. 7C shows a waveform similar to that shown in FIG. 7A but contains a region that has a constant amplitude component between the linearly increasing and decreasing components. These waveforms can be described also in those terms used for FIGS. 3-5 (it ii, and iii) in that they are single continuous waveforms that have regions of higher amplitude for inducing electroporation and lower amplitude regions that can electrically induce the migration of molecules and prolong the electroporated state.

FIGS. 7D-7F indicate three alternative embodiments that use a single linearly increasing component alone or combined with a constant amplitude component in order to provide electromigration either before or after the electroporetic phase of the waveform. FIGS. 7G-71 are similar but contain a linearly decreasing component. FIGS. 7J-7L show rectangular waveforms with at least two regions of constant amplitude each. All of these single continuous waveforms can be described as having regions of higher amplitude for inducing electroporation and lower amplitude regions for producing molecule movement and prolonging the electroporated state.

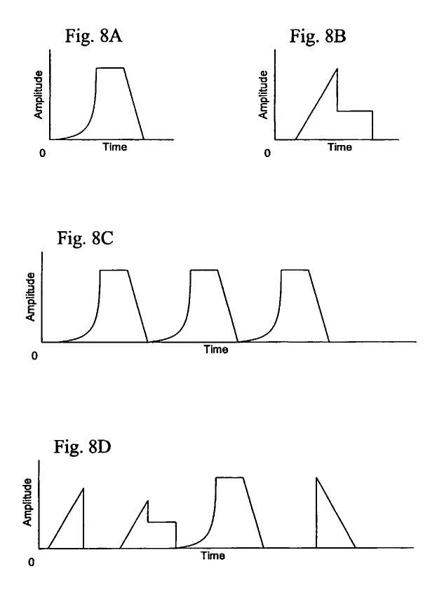

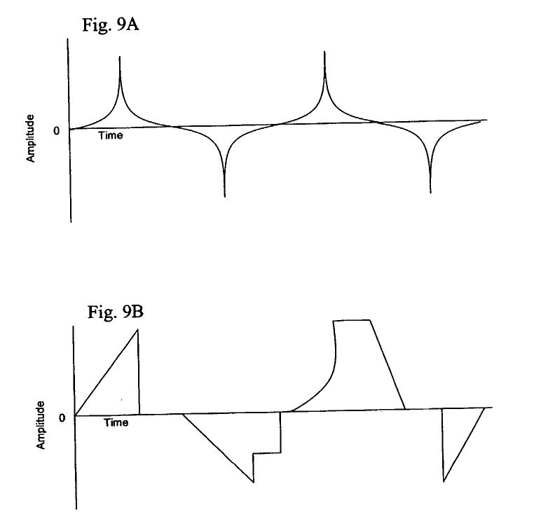

Individual components of the pulses can be combined without departing from the scope of the invention. For example, FIG. 8A shows a waveform with an exponentially increasing, constant amplitude, and linearly decreasing components combined into one continuous signal FIG. 8B shows another alternate embodiment that includes a linearly increasing component followed by a constant amplitude component in one continuous form. The waveforms described above can be combined into pulses which are a series of waveforms that are concatenated. FIG. 8C shows one such pulse that consists of identical waveforms. FIG. 8D shows another type of pulse that consists of more than one waveform. For either type of pulse, the number of waveforms and the time interval between successive waveforms can be the same or different. Further examples of pulses are illustrated in FIGS. 9A and 9B which indicate that some of the waveforms in pulses may have negative amplitudes.

The waveforms know and used in the art relating to this invention have been either rectangular direct current waveforms with positive amplitudes, bipolar rectangular direct current waveforms, and exponentially decreasing waveforms. In addition, pulses consisting of alternating current have been used to effect cell membranes. To the inventors knowledge, these are the only waveforms that have been used to effect cell membranes both in vitro and in vivo. The shape of the waveforms described in this invention differ from those used by others in the field. In addition, they contain components within each continuous waveform that are sufficient to electroporate, cause the electromigration of molecules, or prolong the electroporated state. In constrast, waveforms known in the prior art are separate discrete pulses that are applied with an interval of time between them.

Examples of how to best use the invention for molecule delivery C57BI/6 mice were divided into five treatment groups with four mice per group to demonstrate the invention for performing molecule delivery to muscle cells. A sequence of DNA coding for firefly luciferase contained in a plasmid was injected was injected into the gastrocnemius muscle in the hind limb of each animal. 100 micrograms of the plasmid DNA contained in 50 microliters of liquid were used for each injection. Needle electrodes were inserted into and around the portion of muscle that received the injection. Then, electric pulses were applied to facilitate delivery of the DNA molecules to the interior of the muscle cells. Luciferase expression was then analyzed as evidence of delivery from excised muscle samples 48 hours after delivery of the DNA.

Standard methods were employed to analyze for this commonly used reporter DNA sequence.

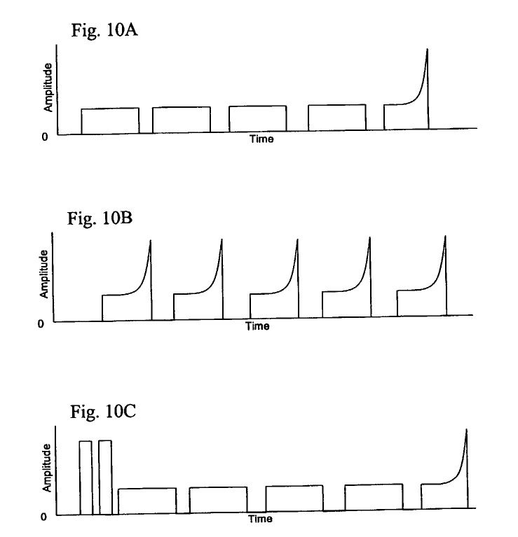

Treatment group 1 received DNA but no electric pulses and served as a control group. Group 2 received a unique series of electrical waveforms for delivery. This waveform series is shown in FIG. 10A. The figure shows four rectangular direct current waveforms that were applied in series. These waveforms had constant magnitudes of 14 Volts/cm (amplitude remained constant for entire pulse duration) which mean that 14 Volts were applied for every cm of distance between electrodes that were of opposite polarity. This terminology is common in the field. The duration of these rectangular waveforlms was 20 milliseconds. A fifth waveform was included as the last waveform in this series. This waveform, as shown in the figure, had a constant magnitude during for a fraction of its total 20 millisecond time duration. However, the latter stages of the waveform had a magnitude that increased with respect to time. These particular fifth waveforms increased with a shape that was approximately exponential. The maximum magnitude of the fifth waveform applied for group 2 animals was 40 Volts/cm. Treatment group 3 received an identical series of waveforms with one difference; the maximum magnitude f the fifth waveform was 100 Volts/cm. Group 4 had yet another maximum magnitude for the fifth waveform applied to the animals. This magnitude was 200 Volts/cm.

A different set of waveforms was applied to deliver DNA to the fifth group. This series was composed of five waveforms that each had constant magnitudes for a period of time after their onset but then exponential I increased in their latter stages. All five of these waveforms had maximum magnitudes that were 200 Volts/cm. The total duration of each waveform was 20 milliseconds. The waveform series applied to group 4 animals is shown in FIG. 10B. Finally, group 5 animals received a series of waveforms s shown in FIG. 10C. These consisted of two high magnitude (750 Volts/cm) short duration (50 microseconds) waveforms followed by two lower magnitude (14 Volts/cm) waveforms that were longer in duration (20 milliseconds). These six waveforms had constant magnitudes during their entire durations. Finally a waveform with an exponentially increasing component was applied at the end of the series with the six pulses. This seventh waveform had a constant magnitude 1 Volts/cm and the maximum magnitude of the exponentially increasing region of the waveform was 100 Volts/cm.

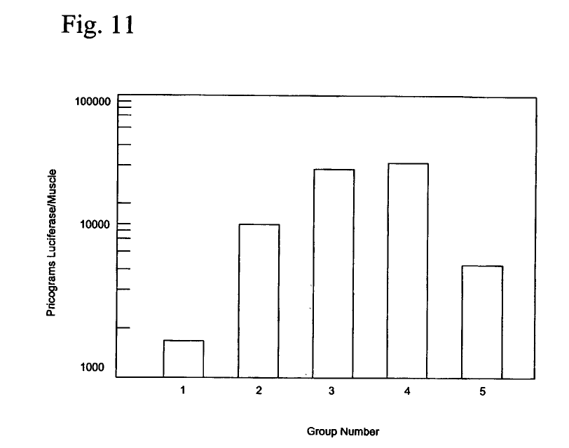

FIG. 11 shows the resulting mean luciferase expression for each of the five animal groups. Results from groups 1 indicate that a certain low level of expression can be attained from simply injecting the plasmid DNA and not administering any electric pulses. The remaining four treatment groups had mean expression that as greater than the expression attained in group 1 indicating that electrical treatment facilitated delivery of the plasmid DNA into the cells. Comparison of the expression from groups 2 and 3 indicates that the fifth waveform maximum magnitude was critical for attaining increased expression as the only difference in the treatment of these two groups was this magnitude. In comparison, electrical treatment for group 4 included only waveforms that had the constant amplitude region followed by an exponentially increasing component. The expression level for group 4 was approximately equal to that of group 3. This further indicates that a series of waveforms that have constant amplitudes followed by an exponentially increasing component can result in high expression. Group 5 used a series of waveforms that were rectangular in shape and experimentally optimized for this application. The expression level for group S was lower than any of the other three groups that received electric pulses to facilitate delivery of the DNA to the interior of the muscle cells. Results of this experiment clearly indicate that using at least one waveform within a series of waveforms that has properties that can cause electromigration and electroporation is beneficial. Electromigration and electroporation are achieved by nature of the change in waveform magnitude over the duration of the waveform. This translates, in the invention, to a one or more component of the waveform applied with an magnitude that is insufficient to cause electroporation but sufficient to cause electromigration of the molecule that is being delivered. It also includes one or more components of the waveform that have sufficient magnitudes to electroporate the cells.

The present invention is an It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1. A method of electromanipulation for effecting substantially simultaneous electroporation and electromigration of molecules into cells by applying to a cellular target a preselected electrical waveform.

2. The method of claim 1 wherein the preselected electrical waveform comprises at least one curved component.

3. The method of claim 2 wherein the at least one curved component has a duration no greater than five minutes and a maximum amplitude no greater than 10,000 V/cm.

4. The method of claim 2 wherein the at least one curved component increases in amplitude as a function of time.

5. The method of claim 2 wherein the at least one curved component decreases in amplitude as a function of time.

6. The method of claim 1 wherein the preselected electrical waveform further comprises increasing and decreasing curved components.

7. The method of claim 6 wherein the preselected electrical waveform further comprises a substantially constant amplitude component interposed between the increasing and decreasing curved components.

8. The method of claim 2 wherein the preselected electrical waveform further comprises a substantially constant amplitude component.

9. The method of claim 8 wherein the substantially constant amplitude component is applied prior to the at least one curved component.

10. The method of claim 8 wherein the substantially constant amplitude component is applied subsequent to the at least one curved component.

11. The method of claim 1 wherein the preselected electrical waveform comprises at least one linear component.

12. The method of claim 11 wherein the at least one linear component has a duration no greater than five minutes and a maximum amplitude no greater than 10,000 V/cm.

13. The method of claim 11 wherein the at least one linear component increases in amplitude as a function of time.

14. The method of claim 11 wherein the at least one linear component decreases in amplitude as a function of time.

15. The method of claim 11 wherein the preselected electrical waveform further comprises increasing and decreasing linear components.

16. The method of claim 15 wherein the preselected electrical waveform further comprises a substantially constant amplitude component interposed between the increasing and decreasing linear components.

17. The method of claim 11 wherein the preselected electrical waveform further comprises a substantially constant amplitude component.

18. The method of claim 17 wherein the substantially constant amplitude component is applied prior to the at least one linear component.

19. The method of claim 17 wherein the substantially constant amplitude component is applied subsequent to the at least one linear component.

20. The method of claim 1 wherein the preselected electrical waveform comprises a plurality of coincident, substantially rectangular components whereby the latest time that the following rectangular component can begin is substantially simultaneously with the completion of the preceding rectangular component.

21. The method of claim 20 wherein the plurality of coincident, substantially rectangular components are of differing amplitudes.

22. The method of claim 20 wherein the plurality of coincident, substantially rectangular components have durations no greater than five minutes and maximum amplitudes no greater than 10,000 V/cm.

23. The method of claim 1 wherein the preselected electrical waveform has an amplitude less than 0.

24. The method of claim 1 wherein the preselected electrical waveform is administered in series.

25. The method of claim 24 wherein at least two preselected electrical waveforms in the pulse are of differing shape.
